(19) United States
(12) Patent Application Publication
(10) Pub. No.: US 2006/0014979 A1
(43) Pub. Date: Jan. 19, 2006

(54) Process for producing terephthalic acid

(76) Inventors: Motoki Numata, Kitakyusyu (JP); Takayuki Isogai, Kitakyusyu (JP); Tomohiko Ogata, Matsuyama (JP)

Correspondence Address: WENDEROTH, LIND & PONACK, L.L.P., 2033 K STREET N. W., SUITE 800, WASHINGTON, DC, 20006-1021, US

(21) Appl. No.: 10/534,913

(22) PCT Filed: Nov. 14, 2003
(86) PCT No.: PCT/JP03/14550

§ 371 Date: Jul. 5, 2005

(30) Foreign Application Priority Data
Nov. 14, 2002 (JP) 2002-330753
Nov. 14, 2002 (JP) 2002-330754

(51) Int. Cl. C07C 63/26

(52) U.S. Cl. 562/410

(57) ABSTRACT

In order to provide a method for producing a high-purity aromatic dicarboxylic acid such as terephthalic acid with high energy efficiency using a simplified process, in the method for producing terephthalic acid according to the present invention, solid-liquid separation and cleaning steps are carried out using a single device, and in a step for removing any liquid adhered to terephthalic cakes by evaporation, internal energy stored in the terephthalic acid cakes and/or the liquid adhered thereto is used at least part of the energy for evaporating the liquid adhered to the cakes.

### TECHNICAL FIELD

The present invention relates to a method for producing an aromatic dicarboxylic acid, particularly terephthalic acid, and more specifically the invention relates to a method for producing a compound obtained by pressurizing and heating in which separation and cleaning steps are carried out using a single common device and which includes the step of utilizing internal energy in removing reaction media and/or cleaning fluids stuck on cakes obtained.

### BACKGROUND ART

Typically, terephthalic acid is obtained in the form of a solid granular product by subjecting a slurry mixture of terephthalic acid and a reaction mother liquor to unit operations of separation and drying.

Many trials have been made before to improve the entire process by improving such unit operations. For example, there are many alternative techniques for solid-liquid separation (as disclosed e.g. in patent document 1). In particular, such devices as horizontal belt filters, rotary vacuum filters and screen bowl decanters (screen bowl centrifuge) have not only a separation function but also have high cleaning capabilities. By using these devices, it is possible to recover a mother liquor, which contains a large amount of impurities, a cleaning filtrate, which is lower in the content of impurities, and cleaned cakes, separately from each other. But such cakes still contain useful liquids. In order to recover such liquids, drying operations or replacement of such liquids with solvents is further needed. Special devices may be needed for separation (as disclosed e.g. in patent document 2).

Examples of drying include drying by external heating with hot gas such as hot air from a compressed air transfer type dryer (as disclosed e.g. in patent document 3). It is also known to obtain solids and gases by evaporating liquids in a slurry in a heated tube (as disclosed e.g. in patent documents 4 and 5). Since these drying operations are separate, independent operations, it is necessary to newly add heat to dry cakes. For this purpose, additional energy and an additional drying device are needed.

It is a general practice to lower the temperature of the slurry while keeping a slurry state for crystallization before solid-liquid separation. For example, it is known to cool the slurry by evaporating the solvent to let the terephthalic acid precipitate (as disclosed e.g. in patent document 6). But evaporation itself serves only to slightly increase the slurry concentration, and has only the effect of lowering the temperature on the process.

It is also known to form slurry again by mixing the terephthalic acid with a cleaning fluid, and flush it (as disclosed e.g. in patent publication 7). While it is well-known to extract powder after forming slurry because it is difficult to extract powder under pressure, a negative aspect of the flushing, i.e. energy loss, has not been recognized. Therefore, the process in which the slurry temperature is lowered by dissipating energy, and the slurry is reheated in the drying step, as disclosed in publications 6 and 7, cannot be said to be energy-efficient.

It is further known to separate cakes under pressure. But it is not suggested that retaining thermal energy before separating the slurry is effective in drying the cakes (see e.g. patent publications 8 and 9).
(Patent document 1: International publication PCT 93/24440; patent document 2: JP patent publication 60-506461; patent document 3: JP patent publication 52-59177; patent document 4: 58-11418; patent document 5: JP patent publication 55-164650; patent document 6: UK patent 1152575; patent document 7: JP patent publication 11-33532; patent document 8: JP patent publication 1-299618; and patent document 9: U.S. Pat. No. 5,698,734)

### DISCLOSURE OF THE INVENTION

As mentioned above, many trials have been made to improve separation and drying operations separately from each other. But no trials have been made to comprehensively improve separation and drying operations as an integrated operation.

An object of the present invention is to provide a method for producing a high-purity aromatic dicarboxylic acid such as terephthalic acid with high energy efficiency using a simplified process.

The inventors have sought ways to achieve this object and discovered that through simple operations of separating and cleaning terephthalic acid cakes with a single common device under a pressure not less than the atmospheric pressure, and continuously using internal energy in removing any reaction medium and/or cleaning fluid adhered to the cakes after cleaning the cakes, it is possible to separately recover a mother liquor, which is high in the content of impurities, a cleaning filtrate, which is low in the content of impurities, and crystals, to which liquid is scarcely adhered. Thus, high-purity terephthalic acid can be produced with high productivity.

The method for producing terephthalic acid according to the present invention includes the below steps (A) to (D), (E) to (I) or (A) to (I). Among these steps, the solid-liquid separation and cleaning steps (B) and (C) and/or (G) and (I) are carried out using a single common device under a pressure not less than the atmospheric pressure. Also, in steps (D) and/or (I), where any liquid remaining in or on terephthalic acid cakes is removed by evaporation, internal energy possessed by the terephthalic acid cakes and/or the liquid adhered to the cakes is used as at least a portion of the energy for evaporating the liquid adhered to the cakes.

(A) Step of oxidizing paraxylene with molecular oxygen in a solvent mainly comprising acetic acid in the presence of a catalyst to obtain terephthalic acid.

(B) Solid-liquid separation step of separating the slurry containing terephthalic acid crystals into crude terephthalic acid cakes and a mother liquor mainly comprising acetic acid.

(C) Step of cleaning the crude terephthalic acid cakes with a cleaning fluid.

(D) Step of evaporating any liquid remaining on or in the crude terephthalic acid cakes after cleaning.

(E) Step of at least partially reducing impurities in crude terephthalic acid by bringing the crude terephthalic acid into contact with hydrogen in a solvent mainly comprising water in the presence of a catalyst.

(F) Step of crystallizing the terephthalic acid in a solvent mainly comprising water by reducing the pressure and temperature of the reaction liquid.

(G) Solid-liquid separation step of separating the slurry into purified terephthalic acid cakes and a mother liquor mainly comprising water.

(H) Step of cleaning the purified terephthalic acid cakes with a cleaning fluid.

(I) Step of evaporating any liquid remaining in or on the purified terephthalic acid cakes.

According to the present invention, through simple operations of separation and extraction, it is possible to separately recover a mother liquor, which is high in the content of impurities, a cleaning filtrate, which is low in the content of impurities, and high-purity terephthalic acid crystals. The present invention is thus industrially valuable in that it saves energy and simplifies the process.

### BRIEF DESCRIPTION OF THE DRAWINGS

FIG. 1 is a flowchart showing a preferred production process according to the present invention.

### BEST MODE FOR EMBODYING THE INVENTION

The present invention is now described in detail.

According to the present invention, in producing terephthalic acid, solid-liquid separation and cleaning are carried out continuously under pressure using a single device which can carry out both of a solid-liquid separation step for obtaining terephthalic acid cakes from a reaction mixture and a step for cleaning the terephthalic acid cakes, such as a screen bowl centrifuge, a rotary vacuum filter, or a horizontal belt filter. Then, after cleaning the terephthalic acid cakes, they are flushed into an atmosphere of which the pressure is lower than the pressure during the cleaning step, thereby using the internal energy of the terephthalic acid cakes and/or the liquid adhered to the cakes to evaporate the liquid adhered to the cakes.

The method for producing terephthalic acid according to the present invention includes two reaction steps, i.e. the oxidation step (A) and reduction step (E). Either step is connected to the solid-liquid separation step and the cleaning step. The solid-liquid separation step and the cleaning step connected to at least one of the steps (A) and (E) are carried out with a single common device, and terephthalic acid cakes produced in these steps are flushed to evaporate any liquid adhered thereto.

Preferably, the solid-liquid separation step and the cleaning step connected to either of the oxidation reaction step and the reduction reaction step are carried out using a single common device, and the terephthalic acid cakes obtained in these steps are flushed to evaporate any liquid adhered to the cakes. In the specification, the steps (A) to (D) are sometimes referred to as crude terephthalic acid (CTA) production steps, and the steps (E) to (H) are sometimes referred to as purified terephthalic acid (PTA) production steps.

[Step (A)]

In Step (A), paraxylene is oxidized with molecular oxygen in a solvent mainly comprising acetic acid in the presence of a catalyst.

Terephthalic acid is an aromatic dicarboxylic acid. According to the present invention, terephthalic acid can be produced by a normal process. Typically, terephthalic acid is produced by reacting paraxylene with molecular oxygen in the presence of catalysts containing heavy metals such as cobalt, iron and manganese, preferably salts of such heavy metals and bromine.

Preferably, a reaction medium used mainly comprises acetic acid. Such an acetic acid solvent is used in an amount 2 to 6 times the weight of paraxylene used. The acetic acid solvent may contain components other than acetic acid, such as water, in such an amount that such other components will not influence the reaction, e.g. 10 percent by weight or less.

The oxidation reaction is typically carried out at a temperature of 130 to 250 degrees Celsius, preferably 150 to 230 degrees Celsius, and at a pressure of 0.2 to 12 MPa, preferably 0.3 to 7 MPa, more preferably 1 to 3 MPa, most preferably 1 to 1.5 MPa.

The reactor used for the oxidation reaction is not particularly limited, and is typically a reactor in the form of a complete mixing tank having an agitator. Reaction is preferably performed continuously. The reaction time (average dwell time) is typically 30 to 300 minutes. The oxidation reaction may be carried out in one stage. But in order to increase the degree of conversion of paraxylene, an additional reactor may be provided in a second stage to carry out oxidation reaction in the additional reactor at a temperature slightly lower than the reaction temperature in the primary reactor, preferably 140 to 190 degrees Celsius. The additional reactor in the second stage may be, besides a reactor in the form of a complete mixing tank, a plug-flow reactor.

The oxidation reaction will convert 95 percent by weight or more, preferably 99 percent by weight or more, of the paraxylene to terephthalic acid, thus producing a slurry in which crystals containing terephthalic acid have separated out. A trace amount of impurities may also be produced. Impurities typically include 4-carboxybenzaldehyde (sometimes referred to as “4-CBA”). Usually, 4-CBA is present in an amount of about 500 to 5000 ppm of the terephthalic acid in the mixture obtained by oxidation reaction.

[Step (B)]

Step (B) is a solid-liquid separation step in which the slurry containing terephthalic acid crystals obtained in step (A) is separated into crude terephthalic acid cakes and a reaction mother liquor.

Step (B) and the subsequent step (C) are preferably carried out using a single common device.

As used herein, crude terephthalic acid refers to cakes that have not yet been subjected to reduction reaction for reducing 4-CBA. Typically, crude terephthalic acid contains 4-CBA by more than 500 ppm of the terephthalic acid.

The reaction mother liquor obtained by solid-liquid separation contains, besides acetic acid as a solvent, water, heavy-metal catalysts, reaction byproducts such as paratoluic acid and 4-CBA, and methyl acetate.

If a screen bowl decanter is used for solid-liquid separation, it is typically operated under a centrifugal force of 500 to 2000 G.

The solid-liquid separator is typically operated under a pressure not less than the atmospheric pressure, preferably not less than 0.2 MPa, more preferably not less than 0.3 MPa, and not more than 22 MPa, preferably not more than 12 MPa, more preferably not more than 7 MPa, particularly preferably not more than 1.5 MPa, most preferably not more than 1.2 MPa. Also, for higher energy efficiency, solid-liquid separation is preferably carried out while maintaining at least part of the pressure during oxidation reaction in step (A).

If it is desired, at the end of step (A), to increase the pressure in the solid-liquid separator for carrying out step (B) than the pressure in step (A), the pressure in the separator is increased with e.g. a pump when transferring the slurry.

[Step (C)]

In step (C), the crude terephthalic acid cakes obtained in step (B) are cleaned with a cleaning fluid under a pressure not less than the atmospheric pressure. As mentioned below, step (C) and the previous step (B) are preferably carried out using a single common device.

The cleaning fluid is not particularly limited and may be an aqueous solvent or an oil solvent. But preferably, it is e.g. a liquid that contains the same acetic acid as the acetic acid as the major component of the oxidation reaction solvent. The acetic acid content is preferably not less than 90%. Instead of acetic acid, an acetate ester, which is relatively low in evaporative latent heat, such as methyl acetate, ethyl acetate, propyl acetate or butyl acetate, may be used.

The pressure in the cleaning step is typically not less than the atmospheric pressure, preferably not less than 0.2 MPa, more preferably not less than 0.3 MPa, and not more than 22 MPa, preferably not more than 12 MPa, more preferably not more than 7 MPa, particularly preferably not more than 1.5 MPa, most preferably not more than 1.2 MPa.

[Single Common Device]

Steps (B) and (C) are preferably carried out using a single common device to reduce the number of devices used for the method according to the invention. Also, by using a single common device, the solid-liquid separation step (B) and the cleaning step (C) can be carried out at equal pressures to each other.

The single common device used for steps (B) and (C) may be a horizontal belt feeder, a rotary vacuum filter, or a screen bowl decanter (screen bowl centrifuge). Among them, a screen bowl decanter is the most desirable. A screen bowl decanter is an integrated device in which both solid-liquid separation and cleaning can be carried out. When the reacted crude terephthalic acid cakes pass through a cleaning station having a filter, a cleaning fluid is sprayed on the cakes, thereby cleaning the cakes. After passing through the filter, the cleaning fluid can be separated from the cakes and recovered.

The screen bowl decanter may be one disclosed in WO98/18750 or WO93/24440. It separates the slurry into solid and liquid under centrifugal force. The solid is transferred on a helical plate to the cleaning station having the filter.

The filter is not particularly limited by its material and shape. For example, it may be a ceramic filter, a wire screen or a metallic bar screen. A desired one is selected taking into consideration the corrosion resistance and the clogging tendency. If a metallic bar screen is used, one which allows a portion of the cakes to pass therethrough should be selected to avoid clogging. By the time the cakes are discharged from the screen bowl decanter, any mother liquor adhered to the cakes is removed. Thus, such cakes are low in the content of impurities.

In a screen bowl decanter, mother liquor and cleaning fluid can be recovered separately from each other. But part of the cleaning fluid may be mixed into the mother liquor. A screen bowl decanter has a high ability to clean the cakes and also withstands commercial use under high pressure.

If a screen bowl decanter is used, the pressure in the solid-liquid separation step (B) is basically equal to the pressure in the cleaning step (C).

[Step (D)]

In step (D), any liquid remaining on or in the cakes obtained in step (C) is removed by evaporation.

The dryer used in the present invention is not particularly limited provided it can perform any of the below-described drying operations, but is typically a pressure dryer having a discharge valve (which is sometimes simply referred to as a “valve”). The discharge valve is not particularly limited as long as it is capable of feeding powder material from a high-pressure side to a low-pressure side. It may be either of the continuous or discontinuous type. For example, it may be one disclosed in WO91/09661. Instead of a single such valve, a plurality of such valves may be used as disclosed in U.S. Pat. No. 5,589,079. A valve or valves disclosed in WO00/71226 or U.S. Pat. No. 4,127,935 may also used.

Upstream of the discharge valve, a cake retaining tank (cake chamber) is usually provided. Cakes cleaned and separated in step (C) are retained in the cake retaining tank. The cakes are then extracted into a powder storage tank by opening the valve. The degree of opening of the valve is preferably controlled so as to always retain a constant amount of cakes in the cake retaining tank.

Operating pressures in the cake retaining tank and in step (C) are essentially equal to each other. The pressure in the powder storage tank is lower than the pressure in the cake retaining tank. By releasing (flushing) pressurized cakes in the cake retaining tank into a lower-pressure atmosphere, the boiling point of liquid adhered to the cakes falls. Due to the fall in the boiling point, sensible heat, i.e. the internal energy stored in the terephthalic acid cakes and/or the liquid adhered thereto, is used to evaporate the liquid adhered to the cakes. Immediately before the cakes are discharged from the cake retaining tank, the temperature of the cakes (TB) is preferably higher than the boiling point (Bp) of the liquid adhered to the cakes in the atmosphere.

Upstream of the valve, the pressure in the dryer is typically kept at not less than the atmospheric pressure, preferably not less than 0.2 MPa, more preferably not less than 0.3 MPa, and not more than 22 MPa, preferably not more than 12 MPa, more preferably not more than 7 MPa, especially preferably not more than 1.5 MPa, most preferably not more than 1.2 MPa.

If a screen bowl decanter is used to carry out steps (B) and (C), the pressures in steps (B) and (C) should be kept at not less than the atmospheric pressure. Terephthalic acid cakes should maintain their pressure in the screen bowl decanter when supplied into the cake retaining tank in step (D). Thus, the pressures in steps (B) and (C) and the pressure in the cake retaining tank in step (D) (i.e. pressures upstream of the valve) should all be kept at not less than the atmospheric pressure, preferably not less than 0.2 MPa, more preferably not less than 0.3 MPa, and at not more than 22 MPa, preferably not more than 12 MPa, more preferably not more than 7 MPa, especially preferably 1.5 MPa, most preferably 1.2 MPa. If the differences between the atmospheric pressure and the pressures in the above respective steps are too small, the internal energy tends to be released insufficiently when flushing, thereby making it difficult to evaporate a sufficient amount of the liquid adhered to the cakes. If these pressures are too high, it may be necessary to increase the pressure resistance of various devices and parts, which pushes up the entire facility cost.

Immediately before being discharged, the temperature of the cakes should be kept at between 50 and 350 degrees Celsius, preferably between 100 and 300 degrees Celsius, more preferably 130 and 250 degrees Celsius. The difference between the boiling point Bp of the liquid adhered to the cakes at the atmospheric pressure and the temperature TB of the cakes immediately before being discharged (i.e. TB minus Bp) is preferably between 5 and 200 degrees Celsius, more preferably between 10 and 150 degrees Celsius, especially preferably between 15 and 100 degrees Celsius.

The interior of the powder storage tank is preferably kept at the atmospheric pressure. But the gas produced when the liquid adhered to the cakes evaporates during flushing will increase the pressure in the powder storage tank to a level slightly higher than the atmospheric pressure. Such gas may be expelled. In such a case, the pressure in the powder storage tank may fall below the atmospheric pressure.

Part of vapor produced in step (D) may be recovered and recycled in steps for producing terephthalic acid. Vapor produced in step (D) mainly comprises vapor of the cleaning fluid that remained on the cakes when the cakes were cleaned with the cleaning fluid. If acetic acid is used as the cleaning fluid in step (C), its vapor, i.e. acetic acid vapor can be advantageously used as a solvent for oxidation reaction in step (A). Such vapor may be directly supplied into the reactor in step (A), or before being supplied to the reactor, it may be condensed in a heat exchanger to recover its thermal energy.

Also, at least a portion of crystals containing terephthalic acid entrained in the vapor produced in step (D) may be recovered and resupplied to a step for producing terephthalic acid. When the crude terephthalic acid cakes are flushed in step (D) to evaporate any liquid adhered to the cakes utilizing the internal energy stored in the cakes and/or the liquid adhered to the cakes, the pressure in the system drops sharply in a short time. This may cause crystals containing terephthalic acid to be entrained in the vapor of the liquid adhered to the cakes. In order to improve the yield of terephthalic acid, such vapor-entrained terephthalic acid crystals are preferably recovered. The crystals thus recovered may be resupplied to any of the CTA and PTA production steps. But since the crystals obtained in step (D) have already been subjected to oxidation reaction, if only the terephthalic acid crystals are recovered by e.g. a bag filter, they are preferably supplied to step (E). More preferably, the terephthalic acid crystals entrained in the vapor produced in step (D) is brought into contact with a liquid mainly comprising acetic acid to obtain a slurry, and the slurry thus obtained is directly or indirectly supplied to step (A) and/or step (B).

[Step (E)]

In step (E), the crude terephthalic acid obtained in step (D) is dissolved in a solvent mainly comprising water to reduce at least part of impurities in the crude terephthalic acid cakes by bringing the CTA into contact with hydrogen in the presence of a catalyst.

In carrying out the reduction reaction, because the crude terephthalic acid is low in solubility at normal temperature, it is necessary to heat it to dissolve it in a solvent mainly comprising water. The reduction reaction is typically carried out at a temperature between 230 and 330 degrees Celsius, preferably between 250 and 310 degrees Celsius. The reduction reaction has to be carried out at a pressure higher than the vapor pressure of the solvent to keep the solvent in a liquid state. The reduction reaction is thus carried out typically at 3 to 12 MPa, preferably at 5 to 10 MPa.

In step (E), 4-CBA contained in the crude terephthalic acid is reduced into paratoluic acid.

[Step (F)]

In step (F), the pressure and temperature of the reaction liquid obtained in step (E) are reduced to crystallize the terephthalic acid in a solvent mainly comprising water.

The crystallization may be conducted continuously or discontinuously. Typically, the crystallization is carried out continuously while reducing the pressure 2 to 6 times, preferably 3 to 5 times in a stepwise manner. During the crystallization step, as a result of flash evaporation of the solvent, the temperature in the system falls. Since paratoluic acid produced by reducing 4-CBA is higher in solubility in water than terephthalic acid, the terephthalic acid will preferentially deposit. But if the pressure is reduced to the atmospheric pressure, the temperature will fall to about 100 degrees Celsius, thereby causing paratoluic acid to crystallize together with the terephthalic acid. Thus, the final crystallizing pressure is preferably not less than 0.2 MPa, more preferably not less than 0.3 MPa, especially preferably not less than 0.5 MPa. Its upper limit is preferably not more than 3 MPa, more preferably not more than 1 MPa, particularly preferably not more than 0.7 MPa. Vapor produced during crystallization may be recovered and recycled in steps for producing terephthalic acid.

[Step (G)]

Step (G) is a solid-liquid separation step in which the slurry containing terephthalic acid crystals obtained in step (F) is separated into purified terephthalic acid cakes and a reaction mother liquor mainly comprising water.

As used herein, “purified terephthalic acid” refers to terephthalic acid in which 4-CBA has been subjected to reduction reaction. Typical purified terephthalic acid cakes contain 4-CBA by not more than 30 ppm of the terephthalic acid.

The solid-liquid separation step is carried out in substantially the same manner at substantially the same pressure range as step (B). But in step (G), the upper limit of the pressure range is especially preferably not more than 1 MPa, and most preferably not more than 0.7 MPa.

The reaction mother liquor produced in the solid-liquid separation contains, besides water as a solvent, a small amount of acetic acid that has been mixed during the previous oxidation reaction, heavy metal catalysts, 4-CBA, which is a reaction byproduct, paratoluic acid obtained by reducing 4-CBA, etc.

[Step (H)]

In step (H), the purified terephthalic cakes obtained in step (G) are cleaned with a cleaning fluid. Except that the cleaning fluid mainly comprises water, this step is the same as step (C).

[Single Common Device]

Steps (G) and (H) are preferably carried out using a single common device, which may be one used for step (C).

A screen bowl decanter, as one of such devices, is an integrated device in which both solid-liquid separation and cleaning can be carried out. When the reacted purified terephthalic acid cakes pass through a cleaning station having a filter, a cleaning fluid is sprayed on the cakes, thereby cleaning the cakes. After passing through the filter, the cleaning fluid can be separated from the cakes and recovered. But part of the cleaning fluid may be mixed into the mother liquor.

The cleaning fluid is not particularly limited and may be an aqueous solvent or an oil solvent. But preferably, it is e.g. a liquid that contains water as its major component like the mother liquor. Since the mother liquor adhered to the cakes is removed by cleaning with a cleaning fluid, the impurity content of the cakes is reduced when the cakes are discharged from the screen bowl decanter. But a portion of the cleaning fluid is adhered to the cakes.

[Step (I)]

In step (I), any liquid remaining on or in the purified terephthalic acid is removed by evaporation. Except that the purified terephthalic acid is dried instead of the crude one, step (I) is identical to step (D).

[Practical Aspect of the Present Invention]

The present invention is characterized in that the mother liquor, cleaning filtrate and crystals with a reduced amount of liquid adhered thereto can be recovered separately from each other through simple operations of separation, cleaning and extraction. Since crystals can be recovered with a reduced amount of liquid adhered thereto, it is possible to omit a separate drier or reduce its size, thereby saving energy. The mother liquor and the cleaning fluid may be recovered without separating them from each other. But since the mother liquor is slightly higher in the impurity content than the cleaning fluid, they are preferably separated from each other and recycled in different steps from each other.

The ability to recover the mother liquor and the cleaning infiltrate separately from each other offers very beneficial, practical advantages to the process. Such advantages are listed below.

At least part of the cleaning fluid (cleaning infiltrate) used to clean the crude terephthalic acid cakes can be recovered and recycled as it is or after being treated in a step for producing terephthalic acid. Since the cleaning filtrate is lower in the impurity content than the separated mother liquor, it can be directly fed into step (A) as a solvent for oxidation reaction. The cleaning infiltrate can also be used as an adsorbent for adsorbing and recovering terephthalic acid crystals entrained in the solvent vapor produced in step (D).

It is also possible to recover crystals containing terephthalic acid from at least part of the cleaning fluid used to clean the crude terephthalic acid cakes, and resupply the thus recovered crystals to a step for producing terephthalic acid. Crystals can be recovered by subjecting the cleaning fluid to solid-liquid separation directly or after promoting crystallization of terephthalic acid by lowering the temperature or pressure. Any ordinary solid-liquid separation method may be used for this purpose, such as centrifugal separation, filtering or precipitation. The recovered crystals can be resupplied to any of the oxidation steps and the hydrogen reduction steps. But since their reaction is substantially complete, they are preferably supplied to the oxidation steps, particularly step (A) and/or step (B).

Further, it is possible to recover at least part of the mother liquor containing acetic acid which has been separated from the crude terephthalic acid slurry by subjecting the slurry to solid-liquid separation, and recycle the thus recovered mother liquor as it is or after being treated in a step for producing terephthalic acid. For example, such mother liquor can be recycled as an oxidation solvent for step (A) because it contains many useful components including oxidation reaction catalysts and reaction intermediates.

It is also possible to recover crystals containing terephthalic acid from at least part of mother liquor mainly comprising acetic acid which has been separated from the crude terephthalic acid slurry by subjecting the slurry to solid-liquid separation, and resupply the thus recovered crystals to a step for producing terephthalic acid. Crystals may be recovered by directly subjecting the mother liquor separated by solid-liquid separation to additional solid-liquid separation, or the separated mother liquor may be subjected to the additional solid-liquid separation after promoting crystallization by lowering the temperature or pressure of the mother liquor. Additional solid-liquid separation carried out after promoting crystallization may be carried out by any ordinary method such as centrifugal separation, filtering or precipitation. The crystals thus recovered may be resupplied to any of the oxidation steps and hydrogen reduction steps, but are preferably supplied to step (A) because an acetic acid solvent is entrained in the crystals.

The mother liquor obtained by solid-liquid separation has a temperature and a pressure that are substantially equal to the temperature and pressure during solid-liquid separation. Preferably, the separated mother liquor is supplied to the oxidation reaction step while maintaining high temperature and pressure. By supplying high-temperature mother liquor to the reactor, it is possible to reduce the energy or sensible heat necessary to heat the oxidation reaction product to a predetermined reaction temperature. This improves energy recovery during oxidation reaction. But if the entire separated mother liquor is recycled, impurities contained in the mother liquor tend to accumulate in the system, thereby deteriorating the quality of the terephthalic acid obtained by solid-liquid separation. Thus, part, preferably 10 to 30%, of the separated mother liquor should be purged to prevent accumulation of impurities. The purged mother liquor contains acetic acid solvents, catalysts for oxidation reaction, and organic impurities derived from impurities in the raw material paraxylene such as benzoic acid. If the mother liquor maintains high temperature, the purged mother liquor may also contain terephthalic acid contained in the separated mother liquor. The purged mother liquor is condensed to evaporate solvents with high-boiling point components remaining as a residue. The residue contains cobalt components and manganese components as catalyst components, and organic impurities. The catalyst components are recovered and recycled in a later step of catalyst recovering and recycling. In order to recover terephthalic acid dissolved in the high-temperature mother liquor, the mother liquor is flushed and condensed by evaporating solvents. When the mother liquor is condensed and cooled by flushing, terephthalic acid will deposit. The deposit is recovered by solid-liquid separation. The solid content thus recovered is preferably supplied to the oxidation reaction step. From the separated liquid that remains after separating the purged mother liquor by solid-liquid separation, active substances such as acetic acid, water and catalysts may be selectively separated and recovered, and the thus separated and recovered substances may be recycled in steps for producing terephthalic acid.

Also, at least part of the separated mother liquor mainly comprising water which is obtained by subjecting the purified terephthalic acid slurry to solid-liquid separation may be recovered and recycled directly or after being treated in a step for producing terephthalic acid. The separated mother liquor may be subjected to solid-liquid separation, distillation or purification using a membrane, and may be recycled as a reduction reaction solvent for step (E).

Further, from at least part of the mother liquor mainly comprising water which has been separated from the purified terephthalic acid, crystals containing terephthalic acid may be recovered, and the thus recovered crystals may be resupplied to a step for producing terephthalic acid. The crystals are preferably recovered by condensing and/or cooling the separated mother liquor to deposit terephthalic acid crystals, and then subjecting the mother liquor to solid-liquid separation. Solid-liquid separation may be carried out by any ordinary method such as centrifugal separation, filtering or precipitation. The crystals thus recovered (secondary crystals) can be resupplied to any of the oxidation steps and the hydrogen reduction steps. But since they are high in the content of intermediates, they are preferably supplied to one of the oxidation steps, particularly step (A). The mother liquor obtained here (secondary mother liquor) can also be directly or indirectly recycled as a reduction reaction solvent for step (E).

Further, at least part of the cleaning fluid (cleaning filtrate) that has been used to clean the purified terephthalic acid cakes may be recovered and recycled directly or after being treated in a step for producing terephthalic acid. Since the cleaning fluid thus recovered is low in the impurity content, it can be advantageously used as a reduction reaction solvent for step (E). The thus recovered cleaning filtrate may be distilled or purified with a membrane. It may also be used as an adsorbent for adsorbing and recovering terephthalic acid crystals entrained in the solvent vapor produced in step (I).

If the cleaning fluid that has been used to clean the purified terephthalic acid cakes contains terephthalic acid that has e.g. passed the filter, crystals containing terephthalic acid may be recovered from at least part of the cleaning fluid by e.g. solid-liquid separation, and resupplied to a step for producing terephthalic acid. The crystals may be recovered by subjecting the cleaning fluid to solid-liquid separation directly or after promoting crystallization by lowering the temperature or pressure. Solid-liquid separation may be carried out by any ordinary method such as centrifugal separation, filtering or precipitation. The crystals recovered may be resupplied to any of the CTA and PTA production steps, but since their reaction is completed, they are preferably supplied to step (F) and/or the separation step (G).

At least part of the vapor produced in step (I) may be recovered and recycled in a step for producing terephthalic acid. The vapor produced in step (I) mainly comprises vapor of the cleaning fluid adhered to the cakes during cleaning in step (H). Thus, if water is used as the cleaning fluid in step (H), steam is produced in step (I). Such steam can be advantageously used as a reduction reaction solvent in step (E). Such vapor may be directly supplied to the reactor in step (E), or before being supplied to the reactor, it may be condensed in a heat exchanger to recover its thermal energy.

At least part of the crystals containing terephthalic acid entrained in the vapor produced in step (I) may be recovered and recycled in a step for producing terephthalic acid. When the purified terephthalic acid cakes are flushed in step (I) to evaporate any liquid adhered to the cakes utilizing the internal energy stored in the cakes and/or the liquid adhered to the cakes, the pressure in the system drops sharply in a short time. This may cause crystals containing terephthalic acid to be entrained in the vapor of the liquid adhered to the cakes. In order to improve the yield of terephthalic acid, such vapor-entrained terephthalic acid crystals are preferably recovered. The crystals recovered may be resupplied to any of the CTA and PTA production steps. But since the crystals obtained have already been subjected to reduction reaction and cleaning, they may be supplied as an end product. The terephthalic acid crystals are preferably recovered in the form of a slurry by bringing them into contact with a liquid mainly comprising water. The slurry thus obtained may be supplied to any of steps (E) to (G).

[Preferred Embodiment]

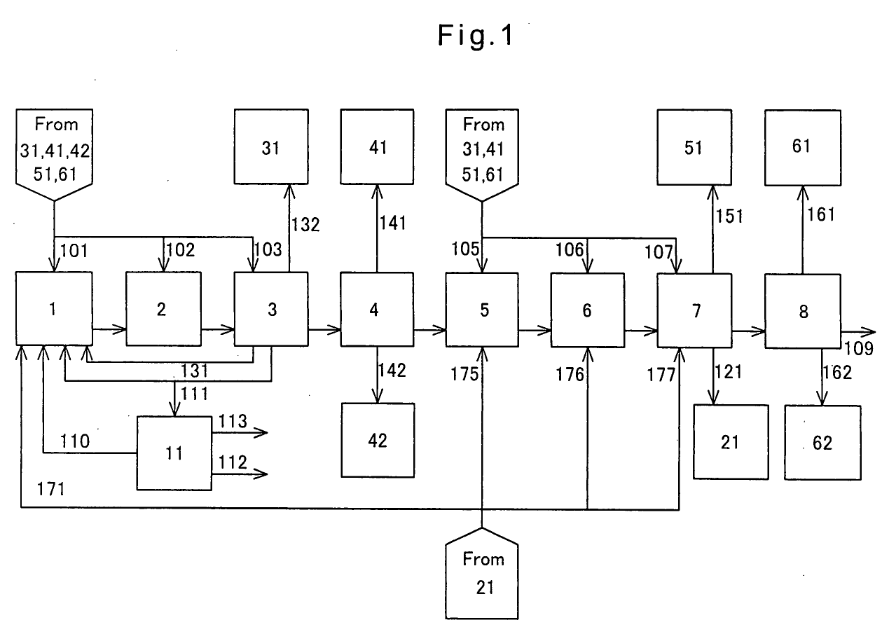

The preferred embodiment of the manufacturing method according to the present invention is now described with reference to FIG. 1.

In FIG. 1, numeral 1 is an oxidation reactor used in step (A); 2, a crystallizing tank; 3, a separator/cleaner for carrying out both steps (B) and (C); and 4, a powder storage tank. Between the separator/cleaner 3 and the powder storage tank 4, a cake retaining tank and a discharge valve (neither is shown) are provided. Crude terephthalic acid cakes obtained in the separator/cleaner 3 are flushed through the cake retaining tank into the powder storage tank 4, where any liquid adhered to the cakes evaporates. Numeral 5 designates a reducing reactor used in step (E); 6, a crystallizing tank used in step (F); 7, a separator/cleaner for carrying out both steps (G) and (H); and 8, a dryer. Between the separator/cleaner 7 and the dryer 8, a cake retaining tank and a discharge valve (neither is shown) are provided. Purified terephthalic acid cakes obtained in the separator/cleaner 7 are flushed into the dryer 8 to evaporate any liquid adhered to the cakes. The dryer 8 also serves as a powder storage tank. Since the liquid adhered to the cakes is mainly water, it will not completely evaporate simply by flushing. The cakes are therefore dried in the dryer 8.

Numeral 11 indicates a solvent recovering system (such as a distillation column). It separates a mother liquor left after oxidation reaction as well as a mixture containing acetic acid supplied from other locations of the process into individual components. The mother liquor contains acetic acid as a solvent, water produced by oxidation reaction, and high-boiling point components such as impurities and oxidation catalysts. Acetic acid can be supplied through line 110 into the oxidation reactor 1. Water is fed through line 112 and discarded or recycled as it is or after being purified as process water. Impurities, which remain at the tank bottom, are discarded after recovering useful components such as catalysts.

Cleaning fluid (mainly comprising acetic acid) used in the separator/cleaner 3 is fed through line 131 into the oxidation reactor after cleaning. If it is desired to separate any terephthalic acid that has passed the filter from the cleaning fluid, the cleaning fluid is fed through line 132 into a solid-liquid separator 31. The thus separated terephthalic acid and acetic acid can be recycled in the process.

When the terephthalic acid cakes are flushed into the powder storage tank, liquid adhered to the cakes evaporates and is gasified with part of the terephthalic acid entrained in the gas. The gas, which contains terephthalic acid crystals, is returned to a liquid state, and fed into a solid-liquid separator 41 to separate it into terephthalic acid and liquid mainly comprising acetic acid. Alternatively, in a solid recovering device 42, the terephthalic acid crystals entrained in the gas are brought into contact with acetic acid to obtain a slurry, the gas is condensed, and the slurry and the condensate liquid are supplied into the oxidation reactor through line 101.

A mother liquor used for reducing reaction which is separated by the separator/cleaner 7 is fed into a solid-liquid separator through line 121. In the solid-liquid separator 21, terephthalic acid and reaction intermediates such as paratoluic acid that deposit by condensing and/or cooling the separated mother liquor are separated and recovered. The remaining reaction mother liquor, which mainly comprises water, is discarded, or purified by evaporation or through a film and recycled as process water. Active substances contained in the water, such as oxidation reaction catalysts or paratoluic acid may be recovered by ion exchange or adsorption.

The cleaning filtrate used in the separator/cleaner 7 (liquid mainly comprising water) is fed to a solid-liquid separator 51 through line 151 after cleaning. In the solid-liquid separator 51, solid contents containing terephthalic acid that has passed the filter and the cleaning fluid are separated from each other. The thus separated terephthalic acid and water can be separately recycled in the process. In this case, the solid contents are preferably returned into the crystallizing tank 6, while the separated cleaning fluid is preferably returned into the reducing reactor 5. The cleaning fluid containing terephthalic acid itself may be recycled in the process without passing it through the solid-liquid separator 51.

When the terephthalic acid cakes are flushed into the dryer 8, liquid adhered to the cakes evaporates and is gasified with terephthalic acid entrained in the gas. The gas containing terephthalic acid is condensed into a liquid, which is fed to a solid-liquid separator 61 and separated into terephthalic acid and liquid that has been adhered thereto, which mainly comprises water. Alternatively, in a solid recovering device 62, terephthalic acid crystals entrained in the gas are brought into contact with water to obtain a slurry, and the gas is condensed into a liquid. The slurry and the condensed liquid can be supplied to any of the reduction reactor 5, crystallizing tank 6 and separator/cleaner 7.

Any of the solid-liquid separators 21, 31, 41, 51 and 61 may be provided with a cleaner. Solid and liquid that have been separated from each other, or the solid alone or the liquid alone, can be resupplied to any step for producing terephthalic acid through lines 101, 102, 103, 105, 106, 107, 171, 175, 176 and/or 177. The solid and liquid may be mixed together again. Also, the solid-liquid mixture may be directly resupplied to any of the steps for producing terephthalic acid through lines 101, 102, 103, 105, 106, 107, 171, 175, 176 and/or 177 while bypassing the solid-liquid separation step. Part of it may be discarded too. An end product is discharged through line 109.

### EXAMPLES

Examples of the present invention are now described. It is to be understood that the present invention is not limited to the examples.

Paraxylene, acetic acid in an amount of 5.5 times the paraxylene in weight, and cobalt acetate, manganese acetate and hydrogen bromide as catalysts were supplied into a liquid-phase oxidation reactor in a plant capable of producing terephthalic acid at a rate of 39 tons per hour, and subjected to oxidation reaction at a temperature of 197 degrees Celsius and at a pressure of 1.45 MPa for 90 minutes (average dwell time). The catalysts were used in such amounts that the total cobalt metal content will be 280 ppm by weight of the solvent, the total manganese content will be 280 ppm by weight of the solvent, and the total bromine content will be 700 ppm by weight of the solvent.

As a gas for carrying out oxidation reaction with molecular oxygen, air was used. The oxygen content of the air was 21%. Compressed air was supplied into the reactor so that the oxygen content of gas discharged from the reactor (which is hereinafter sometimes referred to as waste gas) will be 5 volume percent. Then, oxidized slurry was continuously transferred to a low-temperature additional oxidation reactor, and air (oxygen content: 21%) as the gas for oxidation reaction was supplied to carry out low-temperature additional oxidation at a temperature of 190 degrees Celsius, at a pressure of 1.3 MPa for 35 minutes (average dwell time) so that the oxygen content in the waste gas will be 6% by volume.

The slurry obtained by the low-temperature additional oxidation reaction was carried out continuously in a three-stage intermediate treatment tank, subjected to solid-liquid separation at the atmospheric pressure, and crude terephthalic acid granules were dried with a dryer using vapor as a heat source.

The thus dried terephthalic acid granules were turned into water slurry, which was then purified by adding hydrogen in a reducing atmosphere of 280 degrees Celsius and 8 MPa. Then, it was subjected to continuous crystallization. In the final crystallization tank, the pressure was reduced to 0.62 MPa and the temperature was reduced to 160 degrees Celsius.

### Example 1

The slurry containing purified terephthalic acid obtained in the above manner was introduced into a screen bowl decanter (screen bowl centrifuge), which is an integrated separator/cleaner, and then the cakes were passed through a flushing valve (discharge valve). The flush valve used was one as disclosed in WO91/09661. The pressure in the cake retaining tank, which is provided upstream of the valve, was 0.64 MPa with the powder storage tank, which is provided downstream of the valve, open to the atmospheric pressure. Every time, the valve was opened for one second to discharge 23 kg of cakes. The slurry was supplied to the screen bowl decanter at a rate of 4.5 tons per hour with the cleaning fluid (water) supplied at a rate of 2.0 tons per hour.

The mother liquor separated in the centrifuge contained 900 ppm of impurities. The cleaning liquid used to clean the separated cakes contained 240 ppm of impurities. The discharged cakes contained 115 ppm of impurities. Their liquid content (weight of the liquid adhered to the cakes/dry weight of the cakes) was 4.3 percent.

### Reference Example 1

Tests were conducted under the same conditions as in Example 1 except that no cleaning fluid was run. The discharged cakes contained 160 ppm of impurities.

### Reference Example 2

On the assumption that the mother liquor and the cleaning fluid were not recoverable separately from each other, the content of impurities in the recovered liquid was inferred to be 590 ppm.

### Reference Example 3

On the assumption that no flushing valve was used, the liquid content if the internal energy was not usable for evaporation of the liquid adhered to the cakes was inferred from heat balance to be 8.8 percent.

[Results]

When Example 1 is compared with Reference Example 1, it can be seen that the screen bowl decanter plays an extremely important role in cleaning the cakes. When Example 1 is compared with Reference Example 2, it can be seen that by independently recovering the cleaning fluid, it is possible to advantageously use the cleaning fluid, which is low in the content of impurities. When Example 1 is compared with Reference Example 3, it can be seen that by using internal energy, it is possible to save much energy. These advantages are obtained by using the screen bowl decanter together with internal energy.

### Example 2

The terephthalic acid slurry obtained by low-temperature oxidation was introduced directly into a screen bowl decanter (screen bowl centrifuge) while bypassing the crystallization tank. The pressure in the screen bowl decanter was maintained at around 0.93 MPa. The slurry was supplied at a rate of 20 tons per hour, while the cleaning fluid (acetic acid) was supplied at a rate of 18 tons per hour.

In the screen bowl decanter, the slurry was separated into cakes and a mother liquor by solid-liquid separation. The cakes were cleaned with a cleaning fluid (acetic acid). The thus cleaned cakes were supplied into the cake retaining tank with the pressure in the cake retaining tank maintained at about 0.93 MPa. At the bottom of the cake retaining tank, a flushing valve as disclosed in the WO91/09661 was provided. By opening the flushing valve, the cakes stored in the cake retaining tank were transferred to the powder storage tank, which is open to the atmosphere. When the cakes were transferred from the cake retaining tank to the powder storage tank, the internal energy stored in the liquid adhered to the cakes and the cakes was released and used as heat for evaporating the liquid adhered to the cakes. After flushing, the liquid content of the cakes (weight of the liquid adhered to the cakes/dry weight of the cakes) was 0.2 percent.

Thus, by separating and cleaning the terephthalic acid slurry under high pressure in the CTA production steps, it is possible to eliminate the necessity of a crystallization tank and a dryer, which were heretofore necessary in the CTA production steps. This in turn simplifies the entire plant.

### Example 3

In Example 2, the reaction mother liquor was separated and recovered in the screen bowl decanter at a temperature of 185 degrees Celsius and at a pressure of 0.93 MPa. By purging 20 percent by weight of the reaction mother liquor, it was possible to recycle the remaining portion of the mother liquor in the oxidation reactor.

That is, by separating and cleaning the terephthalic acid slurry under high pressure, it was possible to effectively use the energy stored in the reaction mother liquor as energy necessary for oxidation reaction without loss.

### Example 4

In Example 2, when the cakes were flushed, liquid adhered to the cakes, which mainly comprised acetic acid, mostly evaporated at a rate of 2 tons per hour. Dried cakes were partially entrained in the evaporated acetic acid gas. The acetic acid vapor was therefore introduced into a solid recovering device from its bottom through a pipe, and liquid-state acetic acid was supplied from the top of the solid recovering device by spraying to bring the sprayed acetic acid into contact with the terephthalic acid entrained in the acetic acid vapor, thereby obtain a slurry. The slurry obtained was directly supplied to the oxidation reaction step.

The acetic acid vapor that has passed the solid recovering device contained a small amount of methyl acetate as a byproduct. Thus, by recovering the acetic acid and methyl acetate in the vapor and supplying them to the oxidation reaction step, it was possible to reduce the loss of acetic acid solvent.

1. A method for producing terephthalic acid comprising:
(A) a step of oxidizing paraxylene in a solvent mainly comprising acetic acid in the presence of a catalyst with molecular oxygen to obtain a slurry containing terephthalic acid crystals;
(B) a solid-liquid separation step of separating said slurry into crude terephthalic acid cakes and a mother liquor mainly comprising acetic acid under a pressure not less than the atmospheric pressure;
(C) a step of cleaning said crude terephthalic acid cakes with a cleaning fluid under a pressure not less than the atmospheric pressure; and
(D) a step of evaporating any liquid remaining on or in said crude terephthalic acid cakes after cleaning;
characterized in that:
said steps (B) and (C) are carried out using a single common device; and
internal energy possessed by said terephthalic acid cakes or liquid remaining on or in said terephthalic acid cakes is used as at least a portion of the energy for evaporating the liquid remaining in or on said terephthalic acid cakes in said step (D).

2. A method for producing terephthalic acid comprising:
(E) a step of at least partially reducing impurities in crude terephthalic acid by bringing said crude terephthalic acid into contact with hydrogen in a solvent mainly comprising water in the presence of a catalyst;
(F) a step of crystallizing said terephthalic acid in a solvent mainly comprising water by reducing the pressure and temperature of the reaction liquid to produce a slurry containing terephthalic acid crystals;
(G) a solid-liquid separation step of separating said slurry into purified terephthalic acid cakes and a reaction mother liquor mainly comprising water under a pressure not less than the atmospheric pressure;
(H) a step of cleaning said purified terephthalic acid cakes with a cleaning fluid under a pressure not less than the atmospheric pressure; and
(I) a step of evaporating any liquid remaining in or on said purified terephthalic acid cakes;
characterized in that:
said steps (G) and (H) are carried out using a single common device; and
internal energy possessed by said terephthalic acid cakes or liquid remaining on or in said terephthalic acid cakes is used as at least a portion of the energy for evaporating the liquid remaining in or on said terephthalic acid cakes in said step (I).

3. A method for producing terephthalic acid comprising:
(A) a step of oxidizing paraxylene in a solvent mainly comprising acetic acid in the presence of a catalyst with molecular oxygen to obtain a slurry containing terephthalic acid crystals;
(B) a solid-liquid separation step of separating said slurry into crude terephthalic acid cakes and a mother liquor mainly comprising acetic acid under a pressure not less than the atmospheric pressure;
(C) a step of cleaning said crude terephthalic acid cakes with a cleaning fluid under a pressure not less than the atmospheric pressure;
(D) a step of evaporating any liquid remaining on or in said crude terephthalic acid cakes after cleaning;
(E) a step of at least partially reducing impurities in crude terephthalic acid by bringing said crude terephthalic acid into contact with hydrogen in a solvent mainly comprising water in the presence of a catalyst;
(F) a step of crystallizing said terephthalic acid in a solvent mainly comprising water by reducing the pressure and temperature of the reaction liquid to produce a slurry containing terephthalic acid crystals;
(G) a solid-liquid separation step of separating said slurry into purified terephthalic acid cakes and a reaction mother liquor mainly comprising water;
(H) a step of cleaning said purified terephthalic acid cakes with a cleaning fluid; and
(I) a step of evaporating any liquid remaining in or on said purified terephthalic acid cakes;
characterized in that:
said steps (B) and (C) are carried out using a single common device; and
internal energy possessed by said terephthalic acid cakes or liquid remaining on or in said terephthalic acid cakes is used as at least a portion of the energy for evaporating the liquid remaining in or on said terephthalic acid cakes in said step (D).

4. A method for producing terephthalic acid comprising:
(A) a step of oxidizing paraxylene in a solvent mainly comprising acetic acid in the presence of a catalyst with molecular oxygen to obtain a slurry containing terephthalic acid crystals;
(B) a solid-liquid separation step of separating said slurry into crude terephthalic acid cakes and a mother liquor mainly comprising acetic acid;
(C) a step of cleaning said crude terephthalic acid cakes with a cleaning fluid;
(D) a step of evaporating any liquid remaining on or in said crude terephthalic acid cakes after cleaning;
(E) a step of at least partially reducing impurities in crude terephthalic acid by bringing said crude terephthalic acid into contact with hydrogen in a solvent mainly comprising water in the presence of a catalyst;
(F) a step of crystallizing said terephthalic acid in a solvent mainly comprising water by reducing the pressure and temperature of the reaction liquid to produce a slurry containing terephthalic acid crystals;
(G) a solid-liquid separation step of separating said slurry into purified terephthalic acid cakes and a reaction mother liquor mainly comprising water under a pressure not less than the atmospheric pressure;
(H) a step of cleaning said purified terephthalic acid cakes with a cleaning fluid under a pressure not less than the atmospheric pressure; and
(I) a step of evaporating any liquid remaining in or on said purified terephthalic acid cakes;
characterized in that:
said steps (G) and (H) are carried out using a single common device; and
internal energy possessed by said terephthalic acid cakes or liquid remaining on or in said terephthalic acid cakes is used as at least a portion of the energy for evaporating the liquid remaining in or on said terephthalic acid cakes in said step (I).

5. A method for producing terephthalic acid comprising:
(A) a step of oxidizing paraxylene in a solvent mainly comprising acetic acid in the presence of a catalyst with molecular oxygen to obtain a slurry containing terephthalic acid crystals;
(B) a solid-liquid separation step of separating said slurry into crude terephthalic acid cakes and a mother liquor mainly comprising acetic acid under a pressure not less than the atmospheric pressure;
(C) a step of cleaning said crude terephthalic acid cakes with a cleaning fluid under a pressure not less than the atmospheric pressure;
(D) a step of evaporating any liquid remaining on or in said crude terephthalic acid cakes after cleaning;
(E) a step of at least partially reducing impurities in crude terephthalic acid by bringing said crude terephthalic acid into contact with hydrogen in a solvent mainly comprising water in the presence of a catalyst;
(F) a step of crystallizing said terephthalic acid in a solvent mainly comprising water by reducing the pressure and temperature of the reaction liquid to produce a slurry containing terephthalic acid crystals;
(G) a solid-liquid separation step of separating said slurry into purified terephthalic acid cakes and a reaction mother liquor mainly comprising water under a pressure not less than the atmospheric pressure;
(H) a step of cleaning said purified terephthalic acid cakes with a cleaning fluid under a pressure not less than the atmospheric pressure; and
(I) a step of evaporating any liquid remaining in or on said purified terephthalic acid cakes;
characterized in that:
said steps (B) and (C) are carried out using a single common device;
said steps (G) and (I) are carried out using a single common device; and
internal energy possessed by said terephthalic acid cakes or liquid remaining on or in said terephthalic acid cakes is used as at least a portion of the energy for evaporating the liquid remaining in or on said terephthalic acid cakes in said steps (D) and (I).

6. The method for producing terephthalic acid of any one of claims 1, 3, 4 or 5 wherein said cleaning fluid used in said step (C) contains acetic acid.

7. The method for producing terephthalic acid of any one of claims 1, 3, 4 or 5 wherein at least part of vapor produced in said step (D) is recovered and recycled in a step for producing terephthalic acid as it is or after being treated.

8. The method for producing terephthalic acid of claim 7 wherein at least part of vapor produced in said step (D) is recovered and recycled in said step (A) as it is or after being treated.

9. The method for producing terephthalic acid of any one of claims 1, 3 or 4 wherein at least part of crystals containing terephthalic acid entrained in vapor produced in said step (D) are recovered and the crystals thus recovered are resupplied to a step for producing terephthalic acid.

10. The method for producing terephthalic acid of claim 9 wherein at least part of crystals containing terephthalic acid entrained in vapor produced in said step (D) are recovered and the crystals thus recovered are resupplied to said step (A).

11. The method for producing terephthalic acid of any one of claims 2 to 5 wherein said cleaning fluid used in said step (H) mainly comprises water.

12. The method for producing terephthalic acid of any one of claims 2 to 5 wherein at least part of vapor produced in said step (I) is recovered and recycled in a step for producing terephthalic acid as it is or after being treated.

13. The method for producing terephthalic acid of claim 12 wherein at least part of vapor produced in said step (I) is recovered and recycled in said step (E) as it is or after being treated.

14. The method for producing terephthalic acid of any one of claims 2 to 5 wherein at least part of crystals containing terephthalic acid entrained in vapor produced in said step (I) are recovered and the crystals thus recovered are resupplied to a step for producing terephthalic acid.

15. The method for producing terephthalic acid of claim 14 wherein at least part of crystals containing terephthalic acid entrained in vapor produced in said step (I) are recovered and the crystals thus recovered are resupplied to said step (E) and/or (F).

16. The method for producing terephthalic acid of any one of claims 1 to 5 wherein said single common device or each of said single common devices is a screen bowl decanter.
